(19) United States
(12) Patent Application Publication
(10) Pub. No.: US 2014/0059730 A1
(43) Pub. Date: Mar. 6, 2014

(54) WELDING MASK

(71) Applicant: Dong Geun Kim, Gangwon-do (KR)

(72) Inventor: Dong Geun Kim, Gangwon-do (KR)

(21) Appl. No.: 14/118,029

(22) PCT Filed: May 16, 2012
(86) PCT No.: PCT/KR2012/003838

§ 371 Date: Nov. 15, 2013

(30) Foreign Application Priority Data
May 17, 2011 (KR) 10-2011-0046471

(51) Int. Cl. A61F 9/06

(52) U.S. Cl. CPC A61F 9/06
USPC 2/8.2

(57) ABSTRACT

The present invention relates to a welding mask for protecting the eyes of a user from a strong welding light during welding. More particularly, the present invention relates to a welding mask which has a face protecting plate made of a transparent material to protect the face of a wearer from the heat and flame of welding and to enable the wearer to see a broad portion of the surrounding area of a welding point, and which has a light shielding part the light shielding rate of which is higher than that of said face protecting plate so as to block the strong welding light generated from the welding point. The welding mask consists of a head part to be worn on the head of a wearer; and a face protecting plate installed at the front portion of said head part so as to cover the entire face of the wearer, wherein the face protecting plate is injection-molded with a transparent synthetic resin material so as to block harmful light and flame generated during welding, wherein a portion of said face protecting plate has a light shielding part formed thicker than the face protecting plate so as to have a light shielding rate higher than that of said face protecting plate.

### CROSS-REFERENCE TO RELATED APPLICATION

This application claims the benefit of Korean Patent Application No. 10-2011-0046471, filed on May 17, 2011 in the Korean Intellectual Property Office, the disclosure of which is incorporated herein by reference.

### TECHNICAL FIELD

The present invention relates to a welding mask for protecting eyes from welding light during welding, and more particularly, to a welding mask including a face protecting plate that is made of a transparent material so as to widely see an area near a welded portion while protecting a face from welding heat and flames and a light shielding part that has a light shielding rate higher than that of the face protecting plate so as to block intense welding light produced at the welded portion.

### BACKGROUND ART

In general, a welding mask has been used to protect eyes from harmful ultraviolet light, intense visible light, and infrared light produced during arc welding, gas welding and base-material cutting processes and to protect a face of a welder from burns due to welding light and heat or burns due to a fragment of a heated welding material.

The welding mask includes a mask body for protecting the face, which is made from a nylon-based resin plate or a fiber glass panel, and a light shielding plate made of a dark-colored glass material formed at a middle portion of the mask.

However, since the mask body is made of a transparent material, the welding process is checked only through the light shielding plate. Since the light shielding plate is colored in very dark color in order to block various harmful light rays, it is difficult for the welder to see the outside through the welding mask. Thus, there is a problem in that the welder needs to frequently take off the welding mask during a welding operation in order to see a welded portion.

### DISCLOSURE

### Technical Problem

In order to solve the aforementioned problem, an object of the present invention is to provide a welding mask including a face protecting plate that is made of a transparent or translucent synthetic resin material capable of blocking harmful light so as to widely see an area near a welded portion and a light shielding part that has a thickness thicker than that of the face protecting plate and is integrally injection-molded at a portion for observing the welded portion at the time of injection-molding the face protecting plate.

Further, an object of the present invention is to provide a welding mask including a light shielding lens that is capable of being detachably attached to the portion for observing the welded portion of the face protecting plate.

### Technical Solution

In order to achieve the above object, an embodiment of the present invention provides a welding mask.

The welding mask includes a head part put on a head and a face protecting plate provided on a front side of the head part so as to cover the entire face.

The face protecting plate is injection-molded using a transparent synthetic resin material and is provided to block welding flames and harmful light produced during welding, and a light shielding part, which has a thickness thicker than that of the face protecting plate and has a light shielding rate higher than that of at least the face protecting plate, is formed at a part of the face protecting plate.

Another embodiment of the present invention provides a welding mask.

The welding mask includes a head part put on a head and a face protecting plate provided on a front side of the head part so as to cover the entire face.

The face protecting plate is made of a transparent synthetic resin material, is provided to block welding flames and harmful light produced during welding.

The face protecting plate includes a light shielding part including a light shielding lens that has a light shielding rate higher than that of at least the face protecting plate and that is formed to be detachably accommodated at a part of the face protecting plate so as to block harmful light produced during welding.

A through hole is formed in the face protecting plate, and the light shielding lens is attached to the through hole.

The light shielding part includes a rear-side blocking part that has an outer diameter greater than the through hole and an inner diameter smaller than the through hole and that is attached to an area near the through hole so as to prevent the light shielding lens from being separated from a rear surface of the face protecting plate.

The light shielding part includes a front-side blocking part that has an outer diameter greater than the through hole and an inner diameter smaller than the through hole, that is attached to an area near the through hole so as to prevent the light shielding lens from being separated from a front surface of the face protecting plate, and that has an opened upper portion or a opened lower portion through which the light shielding lens is inserted.

### Effect of the Invention

According to an embodiment of the present invention, it is possible to immediately see an area near a welded portion without taking off a mask by using a transparent face protecting plate. Further, since manufacturing cost is hardly increased by integrally forming a light shielding part for seeing the welded portion at the time of injection-molding the face protecting plate, there is an advantage in terms of price competitiveness.

Further, when the light shielding part is detachably attached to the face protecting plate, there is an advantage in that a light shielding lens is easily replaced with a light shielding lens having a different light shielding rate depending on intensity of welding light.

### DESCRIPTION OF DRAWINGS

FIG. 1 is a diagram illustrating an external appearance of a welding mask according to an embodiment of the present invention.

FIGS. 2 (a), (b) and (c) are a cross-sectional view of a portion A-A′ of FIG. 1.

FIG. 3 is a diagram showing a face protecting plate according to another embodiment of the present invention.

FIG. 4 is an exploded diagram of FIG. 3.

FIG. 5 is a diagram illustrating an attachment structure of a light shielding lens and a protecting plate.

FIG. 6 is a side cross-sectional view illustrating a state where the light shielding lens and the protecting plate are attached.

### BEST MODE

A configuration of the present invention will be described in detail with reference to the accompanying drawings.

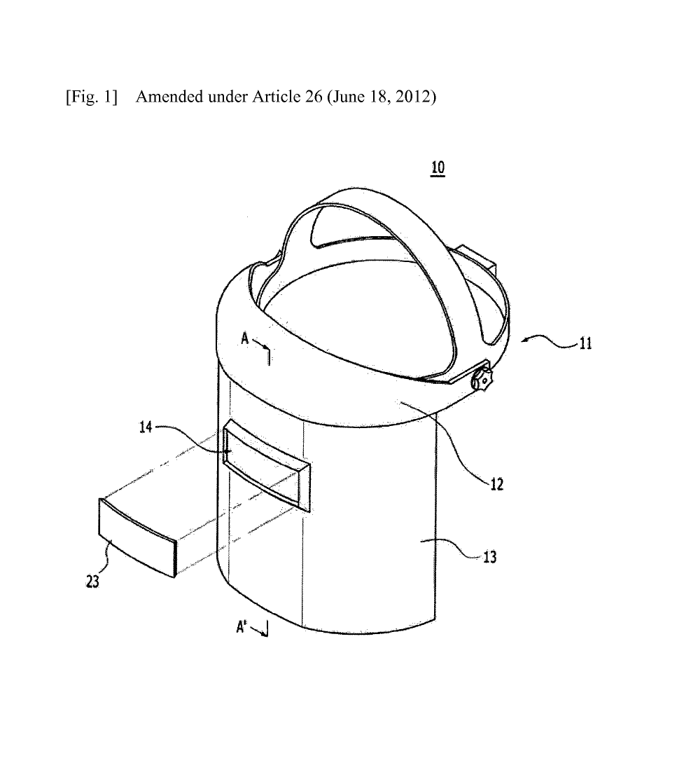

FIG. 1 illustrates an external appearance of a welding mask 10 according to an embodiment of the present invention. A band type head part 11 is put on a head of a welder, a protecting-plate fixing part 12 that rotates in a up and down direction is provided at a front side of the head part 11, and a face protecting plate 13 for protecting a face of the welder by blocking harmful light and welding flames produced during welding is attached to the protecting plate fixing part 12.

The fact protecting plate 13 may have a size capable of covering the face of the welder, and may be injection-molded by using a transparent synthetic resin material colored so as to block welding light. Further, the face protecting plate may be made of high-strength polycarbonate to have high impact resistance.

A light shielding part 14 is integrally formed at a portion (or an area near the portion) of the face protecting plate 13, which corresponds to eyes of the welder, in order to block intense light produced during welding, and the light shielding part 14 is formed to have a thickness thicker than that of the face protecting plate 13 at the time of injection-molding the face protecting plate 13.

Accordingly, the light shielding part 14 is formed to have a shielding rate higher than that of the face protecting plate 13 due to the thickness.

For this reason, the welder can see a welded portion and an area near the welded portion through the face protecting plate 13 having a low shielding rate during a welding operation and can perform the welding operation while directly seeing a portion being welded with the naked eye by blocking bright welding light through the light shielding part 14.

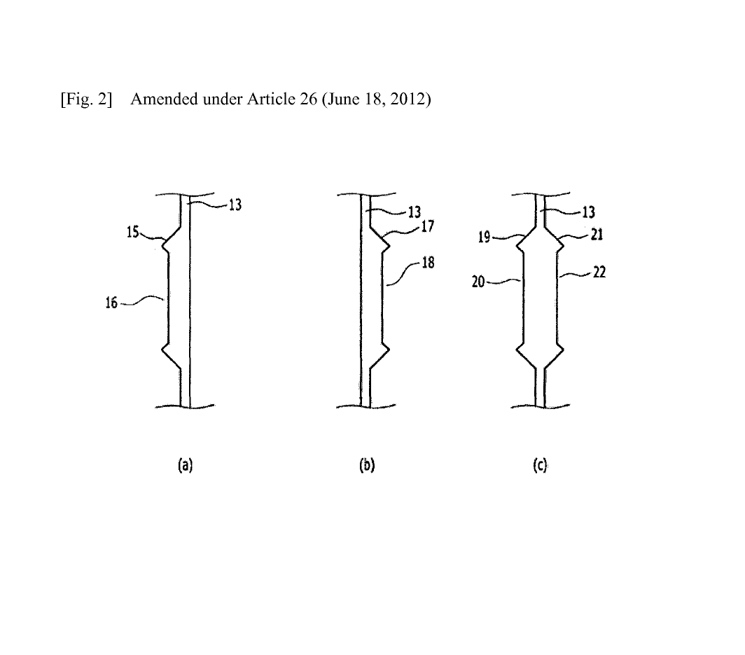

FIG. 2 is a cross-sectional view of a portion A-A′ at which the light shielding part 14 is formed, and (a) of FIG. 2 shows a state where the light shielding part 14 is integrally injection-molded so as to protrude forward from the face protecting plate 13. At this time, a film attachment groove 16 is formed on a front surface of the light shielding part 14 by forming a protrusion 15 so as to further protrude along an edge of the light shielding part 14 that has been formed to protrude.

A transparent protective film 23 is attached to the film attachment groove 16. The protective film 23 prevents the light shielding part 14 from being damaged due to welding flames and is detachably attached to the film attachment groove.

That is, the protective film is made of a synthetic resin material used in a protective film for a liquid crystal display of a cellular phone and may be attached to a smooth surface of the light shielding part 14.

(b) of FIG. 2 shows a state where the light shielding part 14 is integrally injection-molded so as to protrude rearward from the face protecting plate 13, and a film attachment groove 18 by forming a protrusion 17 so as to further protrude at an edge of the light shielding part 14 that has been formed to protrude.

(c) of FIG. 2 shows a state where the light shielding part 14 is integrally injection-molded so as to protrude forward and rearward from the face protecting plate 13. At this time, similarly to the aforementioned examples, film attachment grooves 20 and 22 are formed by forming protrusions 19 and 21 so as to further protrude along an edge of the light shielding part.

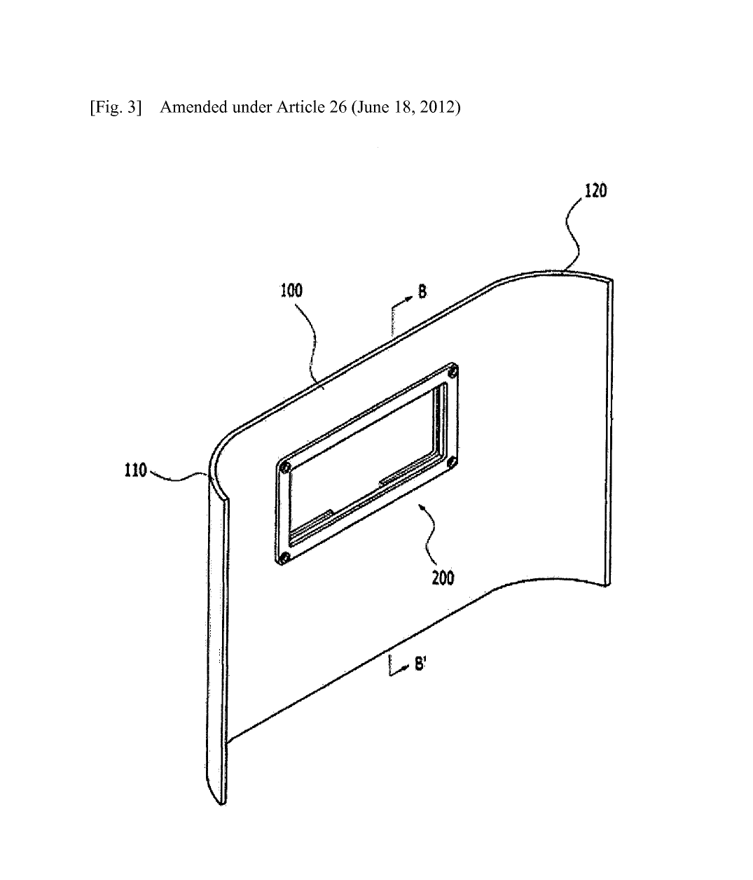

FIG. 3 illustrates s still another embodiment of the face protecting plate 13 according to the present invention, and a plane part 100 and side parts 110 and 120 that are formed by bending both sides of the plane part 100 rearward are integrally injection-molded.

The plane part 100 and the side parts 110 and 120 are made of a transparent synthetic resin material colored so as to block welding light, and are made of high-strength polycarbonate to have high impact resistance.

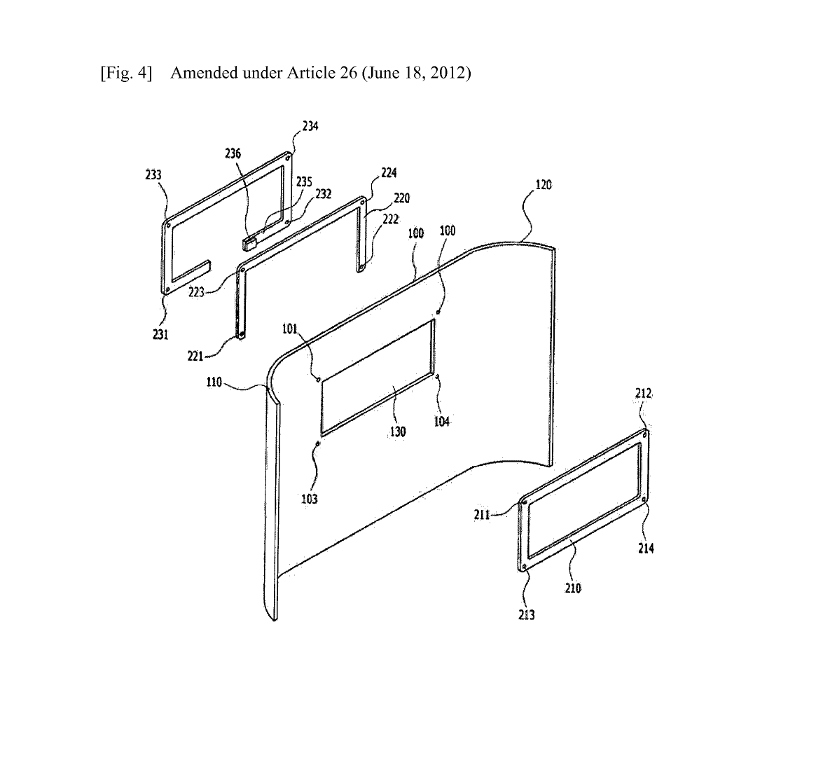

A light shielding part 200 having a light shielding rate higher than that of at least the plane part 100 is formed at an approximately center (preferably, corresponding to the eyes of the welder) of the plane part 100. As shown in FIG. 4, the light shielding part 200 has a structure in which black glass or colored glass of which a light shielding lens is made or a synthetic resin material having a high light shielding rate is accommodated and attached.

In order for the light shielding lens to be attached to a portion where the light shielding part 200 of the plane part 100 is positioned, a through hole 130 is formed so as to have the same shape as that of the light shielding lens, and assembly holes 101, 102, 103 and 140 through which a rear-side blocking part 210, a holder 220 and a front-side blocking part 230 are connected are formed in an area near the through hole 130.

Further, the rear-side blocking part 210 is formed at a rear surface of the plane part 100 to prevent the light shielding lens attached to the through hole from being separated rearward. The rear-side blocking part 210 is formed in a ring shape so as to be provided along an outer circumference of the through hole 130. The rear-side blocking part is connected such that an outer diameter of the rear-side blocking part is greater than the through hole 130 and an inner diameter of the rear-side blocking part is smaller than the through hole 130, so that an inner diameter thereof is positioned inside the through hole 130.

Furthermore, assembly holes 211, 212, 213 and 214 are formed in positions of the rear-side blocking part 210 corresponding to the assembly holes 101, 102, 103 and 104 of the plane part 100.

The holder 220 and the rear-side blocking part 230 are attached to a front surface of the plane part 100 so as to be accommodated in or separated from the top while preventing the light shielding lens from being separated forward. The holder 220 is connected such that an inner diameter of the holder is equal to or greater than the through hole 130 and a lower portion of the holder is open so as to allow the light shielding lens to be inserted from the opened lower portion to be accommodated in or separated, so that the holder does not interfere with the through hole 130.

The opened portion is formed to be equal to or greater than a width of at least the light shielding lens, and assembly holes 221, 222, 223 and 224 are formed in positions corresponding to the assembly holes 101, 102, 103 and 104 of the plane part 100.

The front-side blocking part 230 is connected to a front side of the holder 220, and has a ring shape so as to be provided along the outer circumference of the through hole 130, similarly to the rear-side blocking part 210. The front-side blocking part is connected to the front side of the holder 220 such that an outer diameter of the front-side blocking part is greater than the through hole 130 and an inner diameter of the front-side blocking part is smaller than the through hole 130, so that the inner diameter thereof is positioned inside the through hole 130.

Moreover, the front-side blocking part 230 has a shape in which a part of a lower central portion of the front-side blocking part is open, and an elastic piece 235 can be elastically bent toward by the opened portion. A stopper 236 having a protruding shape is formed inside the elastic piece 235 to prevent the light shielding lens from being separated downward.

In addition, assembly holes 231, 232, 233 and 234 are formed in positions of the front-side blocking part 230 corresponding to the assembly holes 101, 102, 103 and 104 of the plane part 100.

The holder 220 and the front-side blocking part 230 may be integrally formed through injection-molding and attached.

The light shielding part 200 may be connected by being riveted by the aforementioned assembly holes, or may be connected thorough a bolt or bonding.

Further, the holder 220 and the front-side blocking part 230 may be reversely provided in the up and down direction as necessary, and the light shielding lens may be inserted from the top.

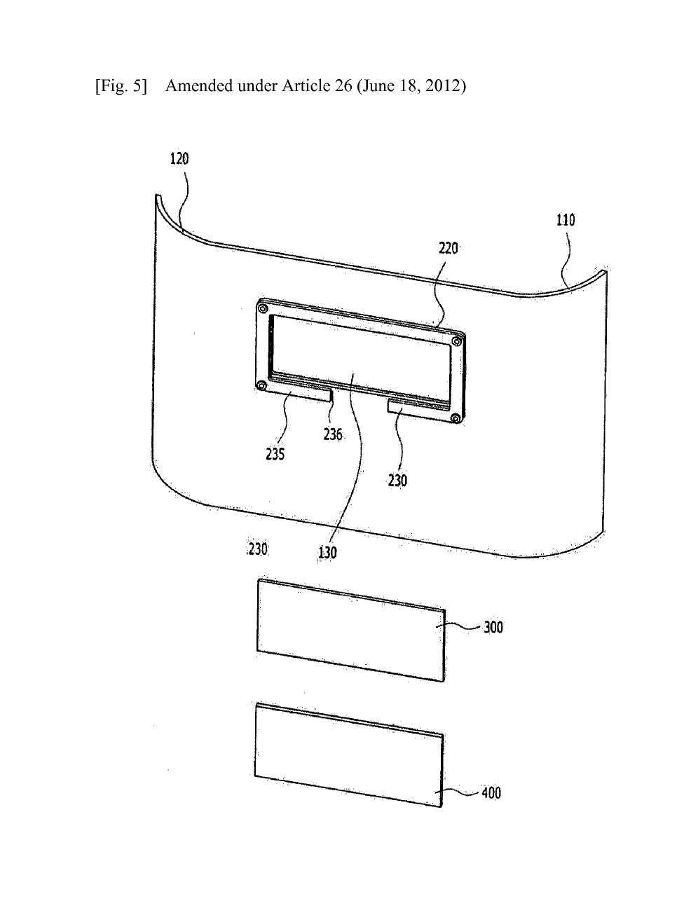

FIG. 5 is a diagram showing an attachment structure of a light shielding lens 300 and a protecting plate 400. At the time of attaching the light shielding lens 300, the elastic piece 235 is pulled forward to be accommodated through the opened lower portion of the holder 220 and the light shielding lens 300 is positioned at the through hole 130.

When releasing the pulling of the elastic piece 235, the elastic piece is restored to an original position. At this time, the stopper 236 comes in close contact with the front surface of the face protecting plate 100 and closes a part of the opened lower portion of the holder 220, so that the light shielding lens 300 is prevented from being separated.

Furthermore, in order for the light shielding lens 300 to be prevented from being damaged due to the welding flames, the protecting plate 400 made from glass or a synthetic resin material such as transparent or translucent polycarbonate is additionally attached, and the protecting plate 400 is attached in the same manner as that of the light shielding lens 300. At this time, the protecting plate is attached to be positioned inside the holder 220.

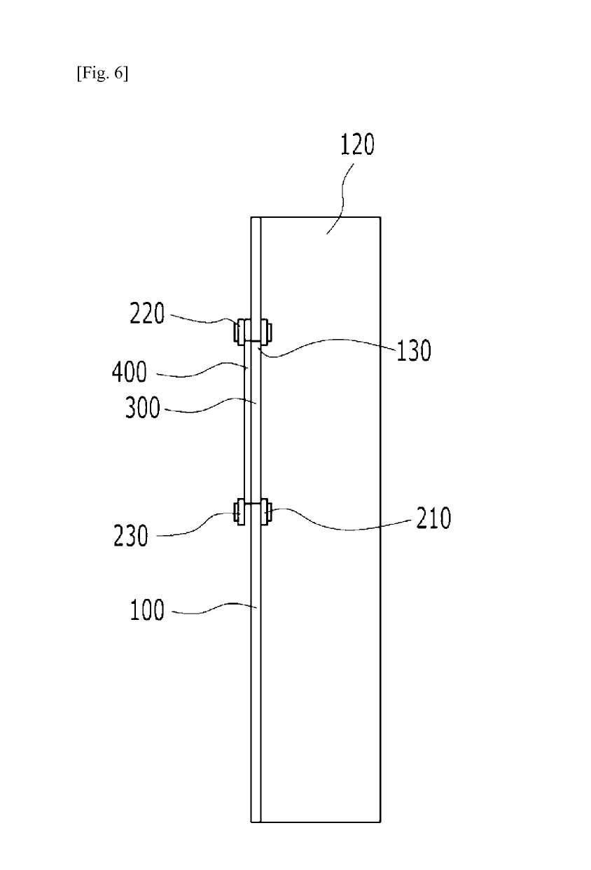

FIG. 6 is a side cross-sectional view of a portion B-B′ shown in FIG. 3. The light shielding lens 300 is positioned inside the through hole 130 and is prevented from being separated rearward due to the rear-side blocking part 210. Further, by inserting the protecting plate 400 into the holder 220 to be attached thereto, the light shielding lens 300 is fixed so as to be prevented from moving toward and the front surface of the light shielding lens 300 is protected from the outside.

To achieve this, it is preferable that the plane part 100 have the same thickness as that of the light shielding lens 300 and the protecting plate 400 have the same thickness as that of the holder 220.

At the time of replacing the light shielding lens 300 and the protecting plate 400, the protecting plate 400 is separated downward by pulling the elastic piece 235, and then the light shielding lens 300 is separated downward by pushing the light shielding lens toward the holder 220. By doing this, the light shielding lens and the protecting plate can be easily replaced.

Accordingly, when the protecting plate 400 is greatly smeared with foreign substances due to the welding flames and the protecting plate needs to be replaced, or when the light shielding lens 300 needs to be replaced depending on intensity of welding light, it is possible to easily replace the protecting plate and the light shielding lens.

1. A welding mask including a head part put on a head and a face protecting plate provided on a front side of the head part so as to cover the entire face, wherein the face protecting plate is injection-molded using a transparent synthetic resin material and is provided to block welding flames and harmful light produced during welding, and a light shielding part, which has a thickness thicker than that of the face protecting plate and has a light shielding rate higher than that of at least the face protecting plate, is formed at a part of the face protecting plate.

2. The welding mask according to claim 1, wherein the light shielding part is formed to protrude forward or rearward from the face protecting plate, or is formed to protrude forward and rearward from the face protecting plate.

3. The welding mask according to claim 2, wherein a protrusion is formed to protrude along an edge of the light shielding part.

4. A welding mask including a head part put on a head and a face protecting plate provided on a front side of the head part so as to cover the entire face,
wherein the face protecting plate is made of a transparent synthetic resin material, is provided to block welding flames and harmful light produced during welding, and includes a light shielding part including a light shielding lens that has a light shielding rate higher than that of at least the face protecting plate and that is formed to be detachably accommodated at a part of the face protecting plate so as to block harmful light produced during welding.

5. The welding mask according to claim 4,
wherein a through hole is formed in the face protecting plate, and
the light shielding lens is attached to the through hole,
wherein the light shielding part includes:
a rear-side blocking part that has an outer diameter greater than the through hole and an inner diameter smaller than the through hole and that is attached to an area near the through hole so as to prevent the light shielding lens from being separated from a rear surface of the face protecting plate; and
a front-side blocking part that has an outer diameter greater than the through hole and an inner diameter smaller than the through hole, that is attached to an area near the through hole so as to prevent the light shielding lens from being separated from a front surface of the face protecting plate, and that has an opened upper portion or a opened lower portion through which the light shielding lens is inserted.

6. The welding mask according to claim 5, wherein a stopper having elasticity is provided at the opened lower portion of the front-side blocking part to prevent the light shielding lens or a protecting plate from being separated upward or downward.
